(19) United States
(12) Patent Application Publication
(10) Pub. No.: US 2026/0248577 A1
(43) Pub. Date: Aug. 27, 2026

(54) A POWERED STEERABLE SYSTEM, A COMPUTER IMPLEMENTED METHOD, AND A COMPUTER PROGRAM PRODUCT FOR NAVIGATING AN ELONGATED SURGICAL DEVICE THROUGH A BODILY LUMEN OF A PATIENT

(71) Applicant: Artiria Medical SA, Borex (CH)

(72) Inventors: Guillaume PETITPIERRE, VEYRIER (CH); Marc BOERS, Borex (CH); Benoît DUBATH, Genève (CH); Loïc SOTTAS, Lausanne (CH); Hadrien MICHAUD, Genève (CH)

(21) Appl. No.: 19/489,696

(22) PCT Filed: Jun. 5, 2023
(86) PCT No.: PCT/EP2023/064959

§ 371 Date: Dec. 3, 2025

(51) Int. Cl. A61B 34/00; A61B 17/00

(52) U.S. Cl. CPC A61B 34/71; A61B 17/00234; A61B 2017/00212; A61B 2017/00309; A61B 2017/00398; A61B 2017/00778

(57) ABSTRACT

The invention relates to a powered steerable system (101) for navigating an elongated surgical device (102) having an outer tubular body (7) and an elongated actuation element (2) nested within the outer tubular body (7) through a bodily lumen of a patient, in particular within the cerebrovascular system. The steerable system (101) comprises: an actuation unit (3) configured to be coupled to the elongated actuation element (2) for actuating the elongated actuation element (2), an input interface which is configured for receiving an input, in particular a user generated input (52), specifying a bent geometric shape (41) of a bendable section (4) at a distal end portion (71) of the outer tubular body (7), a control unit (5) operatively coupled to the input interface and configured for calculating a control command based on the input, and an output interface operatively coupled to the control unit (5) configured for transmitting the control command to the actuation unit (3). The actuation unit (3) is adapted to deflect the bendable section (4) to the bent geometric shape (41) based on the control command.

A powered steerable system, a computer implemented method, and a computer program product for navigating an elongated surgical device through a bodily lumen of a patient The invention relates to a powered steerable system, a computer implemented method, and a computer program product for navigating an elongated surgical device having an outer tubular body and an elongated actuation element through a bodily lumen of a patient, according to the independent claims. The elongated actuation element can e.g. be a tension responsive, a compression responsive element or a combination thereof.

The invention is particularly suitable for performing neurovascular applications which require precise and accurate manoeuvrability within intricate neuronal vasculature of the brain.

Existing elongated surgical devices used in bodily lumens such as intravascular guidewires/catheters often rely on manual manipulation to reshape a distal tip of the elongated surgical device or even necessitate advancement of additional/different surgical devices like guidewires with specifically pre-shaped distal tips.

Various systems for navigating elongated surgical devices in vasculatures known in the prior art are dependent on the skills, in particular manual skills, of a clinician, or are subject of considerable health risks such as vessel damage when manoeuvring through intricate tortuous vasculature.

In particular, in neurovascular applications, the adage “time is brain” emphasizes the importance of rapid surgical intervention which is imperative for minimizing the risks of irreversible brain damage. Reducing the time required for navigation and streamlining the surgical intervention by providing more accurate and efficient navigation of elongated surgical devices is paramount for reducing the overall procedural time.

In particular, the prior art lacks a system for navigating an elongated surgical device to achieve better procedural outcomes and reduced risks to the safety of a patient by allowing for enhanced dexterity and controllability of the elongated surgical device.

WO 2007/008967 A2 discloses a system for controlling the position of an elongated medical device by using a control handle, a robotic device, and a remote-control mechanism which allows the medical device to be positioned within a body of a patient in a remote-controlled manner. However, the system and elongated medical device is complex, large, and does not allow for precise and reliable reshaping of a distal tip of the elongated medical device within the neuronal vasculature.

WO 2017/033182 A1 discloses a double concentric guidewire which has a first guidewire, a second guidewire nested within the first guidewire, and an adjuster mechanism for displacing the second guidewire with respect to the first guidewire by operation of a manual control handle. However, this system is manually operated which limits efficiency and controllability of the guidewire.

There is generally a lack of a simple and compact system for navigating an elongated surgical device which allows for rapid and precise deflection of a distal tip of the elongated surgical device in a reliable and efficient manner. In addition, the prior art lacks a steerable system which does not deviate from common clinical practice and/or requires a steep operational learning curve.

It is an object of the present invention to overcome these and other disadvantages of the prior art.

The invention provides a powered steerable system for navigating an elongated surgical device having an outer tubular body and an elongated actuation element nested within the outer tubular body through a lumen of a patient, in particular within the cerebrovascular system. The steerable system comprises an actuation unit which is configured to be coupled to the elongated actuation element for actuating the elongated actuation element, in particular for applying a tension and/or compression force to the elongated actuation element. The steerable system comprises an input interface which is configured for receiving an input, in particular a user generated input, specifying a deformed and in particular a bent geometric shape of a bendable section at a distal end portion of the tubular outer body.

The deformed geometric shape is not necessarily a single bend, but can also include multiple bends, or other shapes such as 3D shapes. Instead of a user generated input, it is also conceivable to provide an input determined by an automated system, e.g. based on the partly of fully automated analysis imaging data.

The steerable system further comprises a control unit which is operatively coupled to the input interface and configured for generating a control command based on the input. The steerable system comprises an output interface operatively coupled to the control unit which is configured for transmitting the control command to the actuation unit. The actuation unit is adapted to deflect the bendable section to the deformed and in particular the bent geometric shape based on the control command.

The elongated surgical device may comprise or consist of a guidewire or a catheter, preferably a guidewire which has an outer diameter between 0.035″/0.89 mm and 0.010″/0.25 mm, in particular both a guidewire and a catheter. Alternatively, the elongated surgical device may comprise or consist of an interventional device, an implantation device, or a diagnostic device. The tension and/or compression responsive actuation element may comprise or consist of a pull wire, a tendon, or a push rod.

The responsive elongated actuation element and the bendable section of the elongated surgical device may be configured such that the bendable section may be deformed to a first bent geometric shape by applying a compression to the bendable section by retracting/pulling, the responsive actuation element.

Additionally or alternatively, the responsive elongated actuation element and the bendable section may be configured such that the bendable section may be deformed to a second bent geometric shape, in particular opposed to the first bent geometric shape, by applying an extension to the bendable section by extending or pushing the responsive actuation element in a distal direction.

In a preferred embodiment, the actuating unit of the powered steerable system is electrically powered. Alternatively, the actuation unit may be powered hydraulically, pneumatically, magnetically, by ultrasounds or chemically.

If the actuation unit is merely configured to be coupled and not fixedly connected to the elongated surgical instrument, this enables repeated-use capabilities of the actuation unit creating an economically efficient solution by reducing long-term production costs.

The control unit may be configured for calculating at least a first control command and a second control command for deflecting the bendable section to two different bent geometric shapes based on one input. The control unit may further be configured for transmitting the first and second control command to the actuation unit via the output interface in a temporally spaced-apart manner, in particular in a predetermined time interval. It is also possible to have certain geometric shape and the associated commands stored in a memory within the system or in a memory accessible by the system. In this case, the used may simply chose between several predefined configurations, such as e.g. not bent, partly bent or fully bent.

This enables to establish a deflection routine of the bendable section such that the actuation unit is configured to deflect the bendable section to several, e.g. two different bent geometric shapes based on the first and second control commands in a sequential manner. This facilitates the operation of the elongates surgical device and enables for more complex deflection routines without the clinician actively having to prompt additional inputs. In addition, this increases the functionality and versatility of the steerable system by streamlining the surgical procedure and reducing the cognitive and manual demands on clinicians.

The actuation unit may be configured to enable bidirectional movement of the elongated actuation element, facilitating both a proximal-directed and distal-directed linear motion of the elongated actuation element. The actuation unit may be formed by a linear actuation unit or a rotary actuation unit which comprises a conversion mechanism operatively connected or connectable to the elongated actuation element. The conversion mechanism is adapted to translate a rotary movement of the rotary actuation unit into a linear movement of the elongated actuation element.

This actuation unit allows for a precise and accurate control of a spatial position of the elongated actuation element based on the input and allows a compact design without compromising the functionality.

The conversion mechanism of the rotary actuation unit may comprise a spool or roller for winding and unwinding the elongated actuation element, thus ensuring a more compact structure while avoiding peak loads on the elongated actuation element.

The control unit and the actuation unit may be positioned within a common housing of the powered steerable system.

This allows to provide a simplified wiring and protection of the control unit and the actuation unit while providing a space-efficient system for navigating the elongated surgical device.

The common housing may have a longitudinal dimension within a range of 0.1 cm to 25 cm, in particular within a range of 3 cm to 30 cm, preferably within a range of 5 cm to 10 cm and a lateral dimension within a range of 0.036 cm to 10 cm, in particular within a range of 0.5 cm to 5 cm, preferably within a range of 0.7 cm to 1.75 cm. The common housing with the control unit and the actuation unit may have a weight of 1 g to 100 g, in particular between 5 g and 50 g and preferably between 10 g and 30 g.

These longitudinal and lateral dimensions and/or low weight allow for cost savings in terms of manufacturing and a compact design. The dimensions of the steerable system therefore provide improved spatial efficiency of the system without obstructing the clinician which may be a valuable commodity in the limited space of operating rooms/operating table. Moreover, this design renders the device more portable and simplifies the ease of use by clinicians.

The powered steerable system may be configured such that the elongated surgical device, in particular together with the actuation unit, may be manually rotatable and/or translationally movable by a clinician. This simplifies the operation and design of the steerable system.

The powered steerable system may have a decoupling unit which is configured for receiving the elongated surgical device, in particular receiving the outer tubular body and elongated actuation element, such that the rotational and/or translational movement of the elongated surgical device is decoupled from the actuation unit. This allows that the elongated surgical device is rotationally and/or translationally movable with respect to the actuation unit. Ball-bearings may be provided for this purpose. A first bearing may be provided on the outer tubular body connected to the housing and a second bearing may be provided on the inner elongated actuation element or a proximal mount of the inner elongated actuation element connected to the actuation unit. The first and second bearings may be coupled such that the outer tubular body and the elongated actuation element can move in unison, in particular in a rotational direction and/or translational direction with respect to the housing/actuation unit.

The powered steerable system may have a positioning unit which is controllably coupled to the control unit. The positioning unit is configured for (a) rotating the elongated surgical device, in particular together with the actuation unit, around the central trajectory of the elongated surgical device and/or (b) translationally moving the elongated surgical device, in particular together with the actuation unit, in a distal or proximal direction, based on a movement input, preferably a user generated movement input.

This positioning unit enables precise and accurate orienting and spatial positioning of the elongated surgical device in the vasculature of a patient, while facilitating navigation which does not rely on manual skills of a clinician.

The system may also be adapted to be integrated in commercially available positioning units such as robotic-assisted systems which allow clinicians to control percutaneous vascular interventions, such as the CorPath GRX or the Robocath R-One.

The powered steerable system may have a follower unit. The follower unit is designed to compensate the weight and/or torque acting on the actuation unit and in particular to avoid translational perturbation of elements connected to the proximal side of the elongated surgical device. The follower unit may have at least one bearing member which is configured for supporting the actuation unit while moving the actuation unit in the distal, the proximal, and/or a rotational direction around a central trajectory of the actuation unit, when moving the elongated surgical device. Alternatively or additionally, the follower unit may have a sensor, in particular a force or position sensor, which is configured to generate force or position data by detecting a rotational or translational actuation of the elongated surgical device and a follower drive which is adapted to move the actuation unit, and in particular the control unit, along the translational and/or the rotational direction in a synchronized manner with the elongated surgical device based on the force or position data in real-time.

This enables enhanced handling while minimizing/mitigating frictional forces when either manually moving the elongated surgical device or moving the elongated surgical device via the positioning unit. In addition, the follower unit enables to relieve the clinician of physical effort for operating the elongated surgical device by effectively neutralizing the weight of the device, allowing the clinician to work fatigue-free for prolonged surgical applications.

The follower unit may have a biasing member, in particular a spring, which is adapted for providing a predefined biasing force to the movement of the elongated surgical device together with the housing. This allows for more haptic feedback when moving the elongated surgical device enabling a more precise positioning.

The at least one bearing member may be configured for supporting the entire common housing when moving the elongated surgical device.

The at least one bearing member may comprise or consist of a linear and/or rotational ball bearing, roller/spherical bearing, sliding bearing, or air bearing for reducing friction when moving the actuation unit and elongated surgical device.

The force sensor and the follower drive enable an adaptive force control for dynamically supporting the movement of the elongated surgical device and actuation unit in real-time to achieve a smoother and more accurate rotary/translational movement of the elongated device and actuation unit in a synchronized manner.

The follower drive may be connected to the actuation unit or even formed by the actuation unit. Alternatively, the follower unit may be configured to be coupled to the actuation unit such that the actuation unit is movable with respect to the follower unit.

The force sensor may be functionally connected or connectable to the outer tubular body or elongated actuation element to detect the rotation or translational actuation of the outer tubular body or elongated actuation element, e.g. actuation applied manually by a clinician.

The control unit may be configured for applying vibrations via the actuation unit or the positioning unit to the elongated surgical device, in particular to the elongated actuation element and/or the outer tubular body, at a frequency between 1 Hz 1000 Hz, preferably at a frequency between 20 Hz-500 Hz, when deflecting the bendable section. The actuation unit, or in particular the positioning unit, is preferably adapted for applying the vibrations by repeatedly moving the elongated surgical device alternately in the proximal and the distal direction of the elongated surgical device.

This enables an enhanced motion transmission to the outer tubular body and/or the elongated actuation element. The force propagation when deflecting the bendable section based on the control command may be impaired or delayed along the elongated surgical device which may be subject of varying stresses and frictional effects induced by conforming to tight curvatures of the vasculature. The stiction/resistance to motion of the elongated surgical device based on factors such as adhesion or static frictional effects may be overcome by these vibrations, in particular the rapid repeated back and forth movement.

The input interface, in particular a user interface may be electrically connected to the control unit for directly receiving the input or configured for receiving the input via a wireless transmission from a remote control. The steerable system may comprise a wireless input transmission unit couplable to the input interface. The wireless input transmission unit is configured for wirelessly transmitting the input to the input interface.

A wireless transmission simplifies the operation and procedural efficiency of the powered steerable system by allowing the clinician to wirelessly operate the deflection of the bendable section. This is particularly advantageous since a clinician operating the steerable system during surgical procedures, e.g. by manually positioning/orienting the elongated device, may simultaneously wirelessly prompt inputs to deflect the bendable section to the bent geometric shape.

The powered steerable system may have at least one deflection sensor configured to generate deflection data by detecting a position of the elongated actuation element and/or a geometric shape of the bendable section. Deflection in this context refers to any type of deformation, i.e. simple bends, but also more complex structures. The deflection sensor may be arranged at or neighbouring the bendable section and directly determine the deflection. It may also be arranged distant to the bendable section and determine the deflection indirectly, e.g. by measuring the position of or a force acting on the elongated element. The control unit is configured (a) to receive the deflection data from the deflection sensor, (b) to determine if the desired geometric shape is achieved based on the deflection data, and (c) to control the actuation unit based on the detected deflection data in a closed-feedback loop to achieve the desired geometric shape of the bendable section in real-time.

This allows to ensure that the elongated actuation element is reliably positioned to achieve a specific deflection of the bendable section.

The deflection sensor may comprise an encoder which is configured for generating the deflection data of the position of the elongated actuation element based on the motion, in particular the linear or rotational motion, of the actuation unit.

The at least one deflection sensor may be selected from at least one of a magnetic deflection sensor, an optical deflection sensor, a capacitive deflection sensor, or a resistive deflection sensor. At least one deflection sensor is preferably located in the actuation unit or in the bendable section.

The magnetic deflection sensor may be formed by a Hall effect based position sensor. The optical deflection sensor may be formed by a time of flight or reflective based position sensor. The resistive sensor may be formed by a strain gauge or polymer membrane based sensor.

The deflection sensor may at least partially be arranged on the actuation unit and/or elongated actuation element to track a linear or angular position with respect to each other.

If the deflection sensor is arranged in the elongated actuation element for detecting the geometric shape of the bendable section, the deflection sensor may comprise or consist of a strain gauge or an optical fiber having a Bragg grating.

A deflection sensor or imaging data allow for determining the geometric shape of the bendable section in a more exact manner and thus enable a more accurate deflection of the bendable section to the bent geometric shape by adjusting the control of the actuation unit via the control unit in the closed-feedback loop.

It is also possible to provide the system with sensors such as a position and/or force sensor for determining a position of or a force acting on the actuation element or on the actuation unit. This allows to control operation of the actuation unit, without necessarily determining the degree of deflection.

The powered steerable system may comprise an energy storage device, in particular arranged within the common housing, for operating the steerable system.

This improves the portability of the steerable system and freedom in arranging the steerable system in an operating room/on an operating table.

The steerable system may comprise a graphical user interface, which is configured to present visual information on a display. The information can be indicative of the bent geometric shape of the bendable section of the elongated surgical device, but also generally on the status of the system, e.g. that the system is “in operation”, “off”, or “out of battery”. The graphical user interface may additionally or alternatively be configured to provide a movable control element, preferably a slider, on the display which has a spatial position that can be adjusted by a user, in particular bidirectionally adjusted by a user, to adjust a degree of the bent geometric shape, in particular continuously adjust a degree of a uniform lateral bending angle over the entire bendable section.

The graphical user interface may be configured to provide a numerical and/or graphical indicator functionally coupled to the movable control element which displays the degree of the bent geometric shape in real time.

The graphical user interface may be configured to transmit, in particular wirelessly transmit, the input based on the spatial position of the movable control element to the input interface of the control unit specifying the bent geometric shape in real time.

The steerable system may be adapted to use the wireless input transmission unit of the system as a display for the graphical user interface.

The graphical user interface enables a simple and reliable and convenient control of the steerable system, visual feedback of the bent geometric shape and simple reshaping/deflection of the bendable section by one handed movement of the control element.

Alternatively, displays or inputs may be formed by hardware components mounted on the housing of the system or on a remote control. In particular, control displays such as LEDs or a LED display can be used to indicate the degree of deflection or the status of the system.

Also, physical means such as e.g. a physical slider, rotating knobs or a lever may be provided for the control of the system. Depending on the specific use, this may be preferred over a graphical input because it provides direct haptic feedback to the user. The user then does not have to continuously watch the display. It is, however, also possible to enhance a graphic input interface with some feedback means, such as e.g. vibrations on a control device.

Another aspect of the invention relates to a computer implemented method for navigating an elongated surgical device having an outer tubular body and an elongated actuation element nested within the outer tubular body through a bodily lumen of a patient. The method comprises (a) receiving an input, in particular a user generated input, via an input interface specifying a desired and in particular a bent geometric shape of a bendable section at a distal end portion of the outer tubular body and (b) calculating a control command based on the input via a control unit. The method comprises (c) transmitting the control command to an actuation unit via an output interface and (d) deflecting the bendable section to the desired geometric shape based on the control command.

The method optionally comprises applying vibrations via the actuation unit or the positioning unit to the elongated surgical device, in particular to the elongated actuation element and/or the outer tubular body, at a frequency between 1 Hz-1000 Hz, preferably at a frequency between 20 Hz-500 Hz.

The method optionally comprises detecting position and/or force data and moving the actuation unit, and in particular the control unit, along the translational and/or the rotational direction in a synchronized manner with the elongated surgical device based on the force and/or position data in real time. This allows to compensate for weight or torsion of the actuation unit.

Another aspect of the invention relates to a computer program product comprising instructions that cause the previously described powered steerable system to perform the previously described steps of the computer implemented method.

The powered steerable system may comprise the elongated surgical device which has the outer tubular body and the tension and/or compression responsive elongated actuation element nested within the outer tubular body. The distal end portion of the outer tubular body has the bendable section which is deformable to the desired geometric shape.

The bendable section may be configured such that it assumes an essentially straight geometric shape if no external force, in particular tension force, is exerted on it.

The system comprising the elongated surgical device e.g. enables a pre-connection between the elongated actuation element and the actuation unit and optimizes the interaction between the often times intricate elongated actuation element by promoting better reproducibility and reducing the risk of defects.

The elongated surgical device may be torsionally stiff such that a rotational force applied to a proximal end of the elongated surgical device is transmitted along a central trajectory of the elongated surgical device to a distal end of the elongated surgical device.

This enables a reliable rotation of the bendable section in a bent geometric shape at the distal end portion while maintaining the bent geometric shape such that the bendable section can be positioned and oriented within the vasculature, in particular intricate brain vasculature, of a patient.

The bendable section may be uniformly laterally deflectable along its longitudinal length and the bent geometric shape may be defined by a uniform lateral bending angle over the entire bendable section. The control unit and the actuation unit may be configured such that the bending angle is adjustable over the entire range from 0° to 540° in particular the entire range from 0° to 270°, preferably the entire range from 0° to 180°.

The uniformity of the bending angle optimizes the ratio of the load within the elongated surgical instrument in relation to the maximum bending of the bendable section. This uniform distribution of stress allows for maintaining the structural integrity even for large ranges of the bending angle which may be required for being advanced through tortuous vasculatures.

One lateral side of the bendable section of the elongated surgical device may have a stress relief section, in particular comprising at least one, preferably a plurality of, circumferential and/or helical cut-outs, so that the bendable section is deflected in a lateral direction of the stress relief section when the tension and/or compression force is applied to the elongated actuation element. The deflection may occur in two lateral directions within a single plane, preferably in exactly one lateral direction.

This stress relief portion enables a reliable and consistent deflectability of the bendable section without any plastic deformations.

One lateral side of the bendable section of the elongated surgical device, in particular opposite to the stress relief portion, may have a reinforcement portion, preferably comprising a reinforcement structure. The reinforcement structure may be integrally formed with the outer tubular body, and is longitudinally rigid, such that applying the tension and/or compression force to the elongated actuation element essentially does not longitudinally affect the length of the reinforcement portion.

This increases the structural integrity of the elongated surgical device and enables a reliable deflectability of the bendable section.

The bendable section may comprise a plurality of stress relief sections and/or reinforcement portions arranged at different longitudinal subsections of the bendable section. This allows the bendable section to be deformable to more complex bent geometric shapes.

A distal end of the elongated actuation element may be connected to a terminal distal tip of the elongated surgical device, which preferably has a rounded shape, and a proximal end of the elongated actuation element is longitudinally movably connected to the actuation unit and the proximal end of the outer tubular body is longitudinally immovably connected with respect to the actuation unit, in particular connected to a housing of the actuation unit. The connection may be in a manner such that a respective rotation is possible.

This allows a secure connection of the elongated surgical device with the actuator and provides an all-in-one solution ensuring straight out of the box usability without a need for complex assembly of the steerable system.

The elongated surgical device may be sized and shaped for use in peripheral interventions, in interventional cardiology, or in neurovascular surgical procedures. The outer tubular body may have a maximum cross-sectional dimension of less than 1 mm, in particular less than 0.6 mm, preferably less than 0.37 mm. The surgical elongated device may have a length between 0.5 m and 4 m, in particular between 1 m and 3.5 m and preferably between 2 m and 3.15 m.

This small size allows for manoeuvring the elongated surgical device in tortuous intricate vasculatures, such as within the cerebrovascular system. At the same time smaller bending radii can be achieved by this cross-sectional dimension of the outer tubular body. The elongated surgical device may further be used for deflecting micro catheters, e.g. projecting them backward via the bendable section being deflected to the bent geometric shape.

The elongated surgical device may comprise a radiopaque element, preferably located at a distal tip of the elongated surgical device.

The radiopaque element allows for real time localization of the distal tip of the elongated surgical device, in particular by fluoroscopy.

The radiopaque element may also extend along the majority or entire bendable section such that the bent geometric shape may be verified via X-ray imaging techniques. The radiopaque element may be configured to uniformly bend with the geometric shape of the bendable section, in particular by the radiopaque element having a coil shape, e.g. a radiopaque element formed by a platinum iridium coil.

The outer tubular body may be formed by a monolithic tubular element, in particular comprising or consisting of stainless steel or nitinol, preferably formed by a nitinol hypotube, e.g. a laser-cut hypotube.

Nitinol provides for enhanced torqueablility and exhibits super-elastic properties which allows for reliable and repeated recovery of its original shape after being deformed to a bent geometric shape.

The invention is now described with reference to certain embodiments and figures which show:

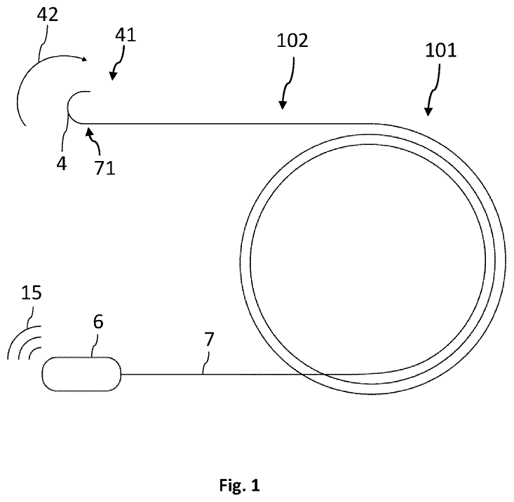

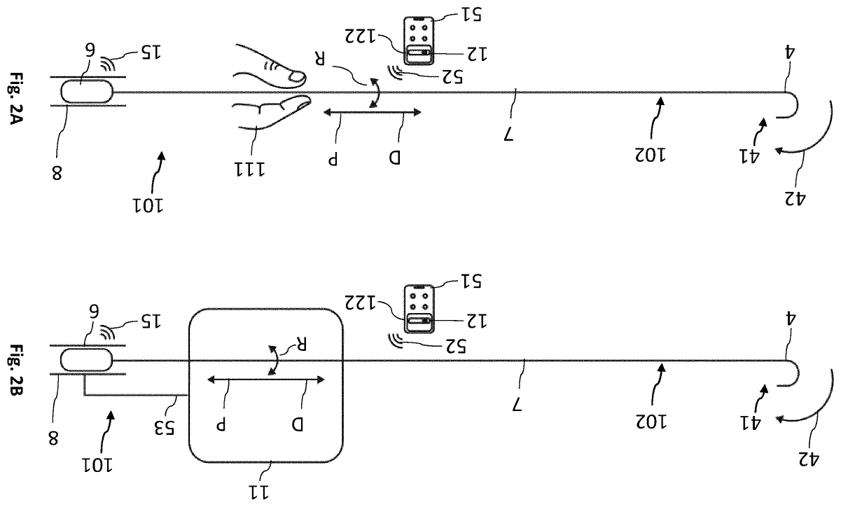

FIG. 1: a schematic plan view of a first embodiment of a powered steerable system for navigating an elongated surgical device by deflecting a bendable section to a bent geometric shape,

FIGS. 2A and 2B: a representation of a second embodiment and third embodiment of the powered steerable system comprising a follower unit and being translationally and rotationally movable by manual operation or a positioning unit respectively,

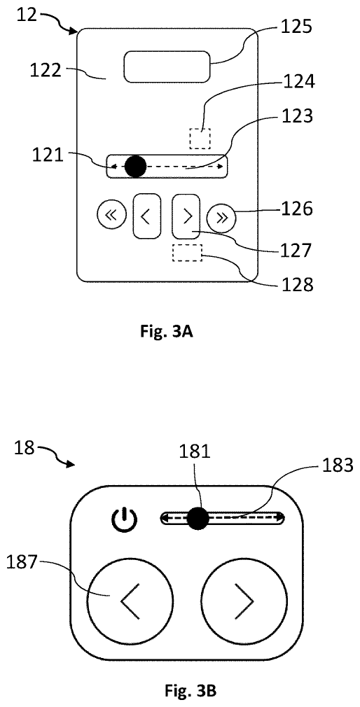

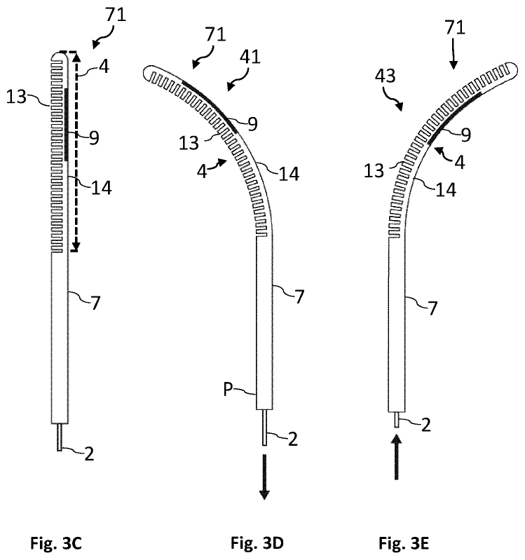

FIG. 3A: a representation of a graphical user interface which is configured to provide a movable control element for adjusting the bent geometric shape based on a user generated input,

FIG. 3B: a representation of a remote control configured to provide a movable control element for adjusting the bent geometric shape based on a user generated input,

FIGS. 3C to 3E: an elongated surgical device having a straight geometric shape, a first bent geometric shape, and a second bent geometric shape respectively,

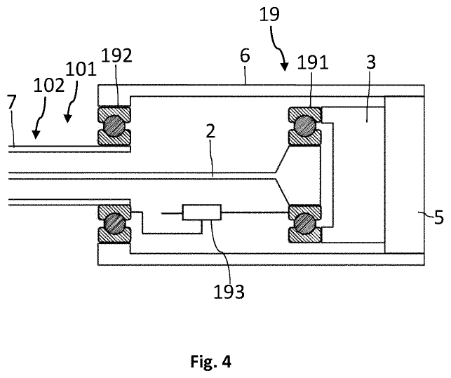

FIG. 4: a cross-sectional view of a powered steerable system having a decoupling unit,

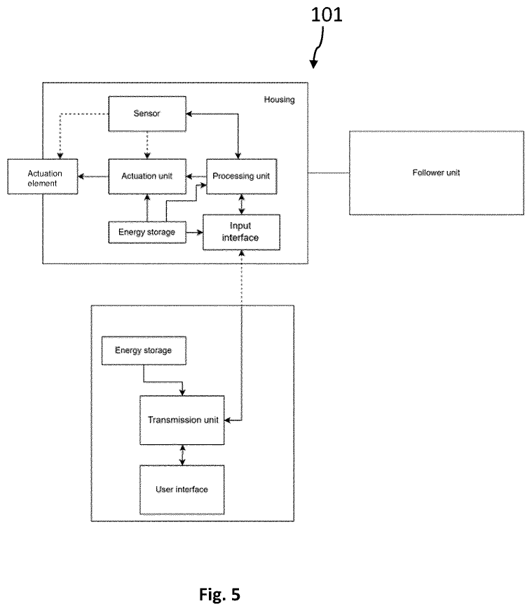

FIG. 5: a schematic view of the various components of the system according to the invention

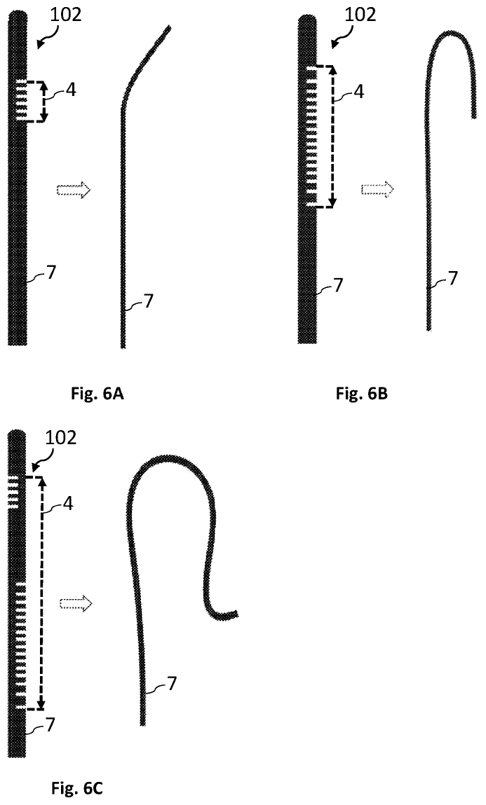

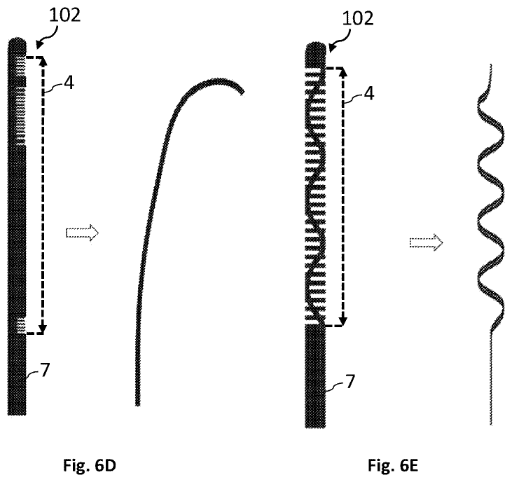

FIGS. 6A to 6E: a schematic view of different embodiments of the elongated surgical device having different reconfigurable tip shapes, including tip shapes which are reconfigurable in 3 dimensions,

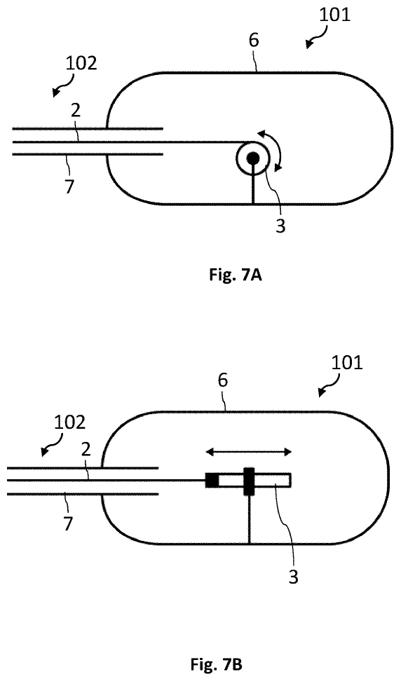

FIGS. 7A and 7B: a schematic view of two different embodiments of an actuation unit,

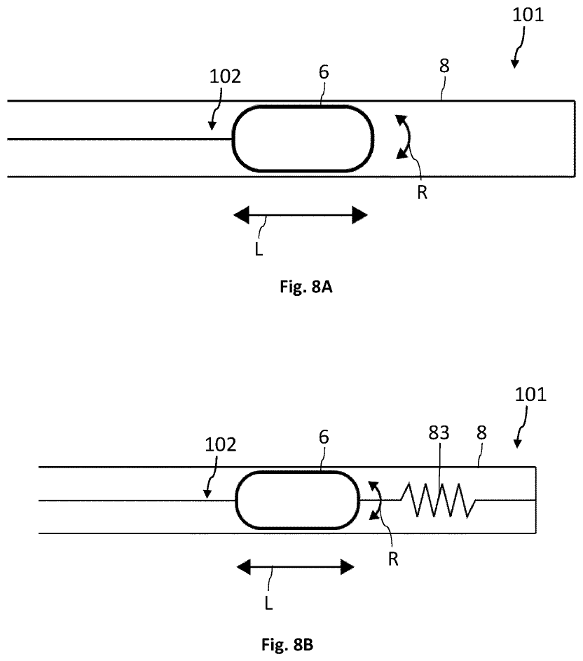

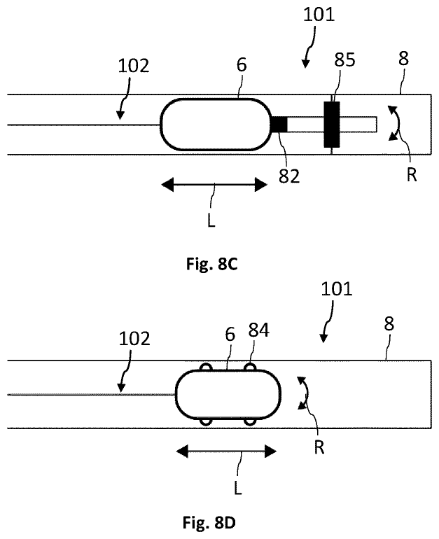

FIGS. 8A to 8D: a schematic view of four different embodiments of a follower unit of the system,

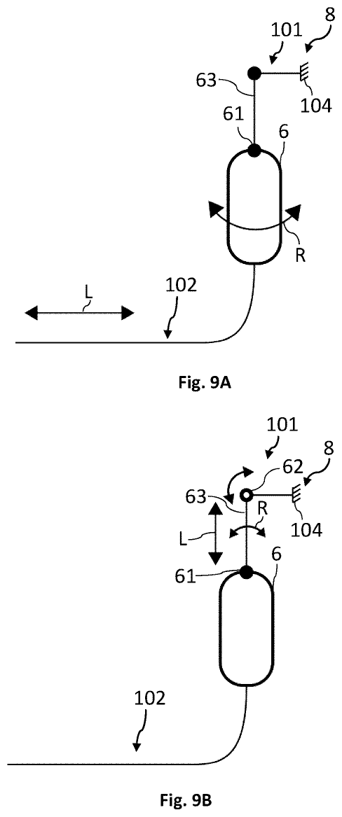

FIGS. 9A to 9B: a schematic view of different embodiments of the follower unit in which the housing is suspended to a solid support,

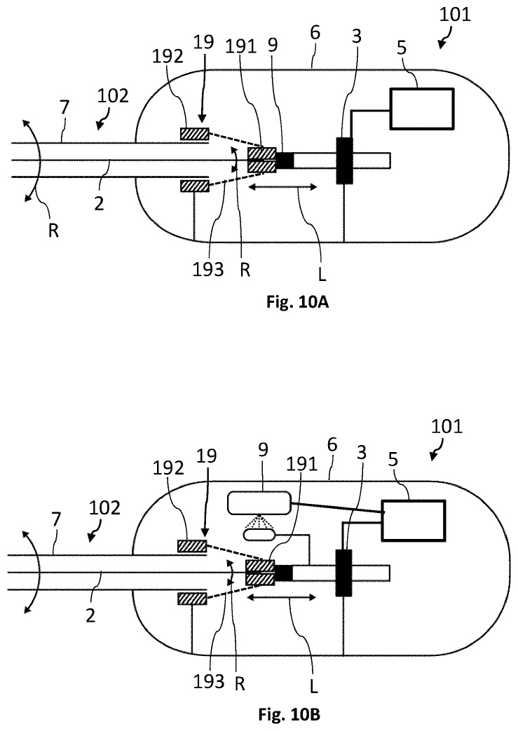

FIGS. 10A and 10B: a schematic view of two different embodiments of a system having two different deflection sensors, and

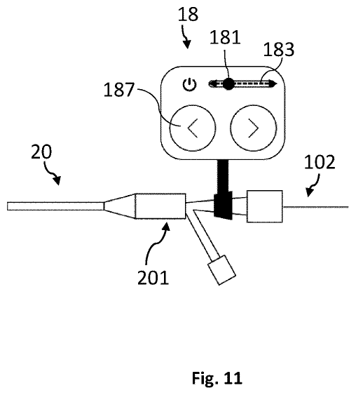

FIG. 11: A wireless input transmission unit according to the invention mounted on a catheter valve connected to a catheter.

FIG. 1 shows a plan view of a powered steerable system 101 for navigating an elongated surgical device 102 formed by a guidewire for neurovascular, peripheral, or cardiac indications.

The powered steerable system 101 has an actuation unit, a control unit, an input interface, and an output interface which are arranged in a common housing 6 at the proximal end of the steerable system 101.

The elongated surgical device 102 has an outer tubular body 7 and at least one, in particular only one, tension and/or compression responsive elongated actuation element formed by a pull wire. The outer tubular body 7 is formed by a tube of metal such as platinum, aluminum, magnesium, gold, stainless steel, titanium, or by a metallic alloy, such as nitinol, cobalt chrome. The tube can also be composed of an assembly of different tube sub-sections, of different metals, joint together through solid joints. Alternatively, the tube can be a single monolithic element, such as a nitinol hypotube which is torsionally stiff. The nominal diameter of the outer tubular body 7 is 0.014″/0.36 mm and has a length between 110 cm and 315 cm, in particular 250 cm. A distal end portion 71 of the outer tubular body 7 has a bendable section 4 which is deflectable based on a user generated input to a bent geometric shape 41. The bent geometric shape 41 which is schematically shown in FIG. 1 has a uniform lateral bending angle over the entire bendable section 4. The bendable section 4 in FIG. 1 is configured to be deflected at the uniform lateral bending angle in a lateral direction 42 but may also be deflectable in three-dimensions. This allows the bendable section 4 to be deflected to a selected bent geometric shape 41 and oriented and advanced/retracted along a trajectory of a branching vasculature or tortuous vasculature.

The input interface is configured for wirelessly receiving a user generated input indicative of the desired bent geometric shape 41 via a wireless transmission 15. The control unit is configured to calculate a control command based on the user generated input and the output interface is configured for transmitting the control command from the control unit to the actuation unit. The actuation unit then longitudinally retracts or advance the pull wire based on the control command such that the bendable section 4 is deflected to the desired bent geometric shape 41.

The control unit is further adapted for applying vibrations at a frequency of 10 Hz via the actuation unit to the elongated actuation element of the elongated surgical device 102 in form of a rapid back and forth movement when deflecting the bendable section. This allows deflecting the bendable section while the motion transmission which is subject to internal stiction may im-pair or delay the deflection of the bendable section 4 to the bent geometric shape 41.

The FIGS. 2A and 2B show a representation of a second and third embodiment of the powered steerable system 101 comprising a follower unit 8 and being translationally and rotationally movable by manual operation (FIG. 2A) or by a positioning unit 11 (FIG. 2B), respectively. The powered steerable system 101 in FIGS. 2A and 2B comprises all previously described elements in FIG. 1 and further has a wireless input transmission unit 51 which is couplable to the input interface for wirelessly transmitting a user generated input 52 indicative of the desired bent geometric shape 41 of the bendable section 4 in a lateral direction 42. The input transmission unit 51 has a graphical user interface 12 which has a display 122 for displaying a movable control element which spatial position can be directionally adjusted by a user to adjust a degree of the bent geometric shape 41. The control unit may be adapted for wireless transmission 15 of status data of the actuation unit, follower unit, and/or the elongated surgical device to the input transmission unit 51, e.g. to display the status data on the input transmission unit 51.

The follower unit 8 in FIGS. 2A and 2B has a bearing member which supports the common housing 6 such that the common housing 6 is movable in a proximal direction P and a distal direction D in unison with the elongated surgical device 102 with minimal frictional resistance. Thus the follower unit 8 enables a more accurate positioning of the elongated surgical device 102 by compensating for the weight/inertia of the common housing 6. The follower unit 8 is optionally further configured for supporting the common housing 6 such that the common housing 6 is movable in a rotational direction R in unison with the elongated surgical device 102 with minimal frictional resistance. However, in a preferred embodiment the rotational movement of the steerable system 101 may have a decoupling unit which is adapted to decouple the rotational movement of the elongated surgical device 102 from the actuation unit/common housing 6, e.g. via circumferential ball bearings for connecting a proximal end of the elongated surgical device 102 and the actuation unit/common housing 6 (see FIG. 4). This allows for a simpler design and does not require a rotary design of the actuation unit/common housing 6 while still facilitating manipulation of the elongated surgical device 102.

The first embodiment of the steerable system 101 in FIG. 2A is manually movable in the distal and proximal direction D, P and in the rotational direction R. This allows a simple design enabling longitudinal and rotational positioning of the elongated surgical device similar to established common practice for clinicians which does not require a high learning curve.

The second embodiment of the steerable system 101 in FIG. 2B is translationally and rotationally movable via the positioning unit 11 which has a longitudinal and rotational actuator. The positioning unit can be formed by a commercially available device, such as devices known provided by e.g. CorPath GRX or Robocath R-One.

The wireless transmission unit 51 is further adapted for transmitting a user generated movement input to the control unit via the input interface. The control unit is coupled to the positioning unit 11 such that the elongated surgical device can be moved in the rotational direction around its longitudinal trajectory or translationally moved in the distal or proximal direction based on the user generated movement input. The control unit may be connected to the positioning unit 11 by a rigid mechanical element 53 as shown in FIG. 2B. Electrical or wireless connectivity of the positioning unit 11 with the positioning unit 11 contributes to increasing the compatibility with commercially available positioning units 11. Motorizing the movement of the elongated surgical device in this manner enhances precision, safety and procedural efficiency.

FIG. 3A shows a representation of a graphical user interface 12 which is configured for providing a movable control element on a display 122 for adjusting the bent geometric shape 141 based on a user generated input. The graphical user interface may be displayed on a wireless transmission unit 51 (see FIGS. 2A and 2B) or may be displayed on an electronic device such as a smartphone or tablet. Alternatively, the graphical user interface 12 may be directly electronically connected to the control unit.

The graphical user interface has a slider 121 which is bidirectionally movable by a user to gradually adjust the deflection of the bendable section 4 in a uniform manner in real time (see FIGS. 3C and 3D). Based on spatial positions 123 of the slider 121, a different user generated input specifying a bent geometric shape of the bendable section is transmitted to the input interface of the powered steerable system. The graphical user interface 12 comprises a numerical indicator 124 which is functionally coupled to the movable slider 121 and displays a per-centage of the total tip actuation/deflection indicative of the bent geometric shape. The display 122 may be configured to receive touch-sensitive input commands by a user and/or have control buttons 126, 127 which can be adapted for selectively fine or coarse adjustment of the spatial position 123 of the slider 121 as shown in FIG. 3A. This allows for a rapid adjustment of the desired bent geometric shape while also allowing an accurate adjustment of the deflection in an intuitive and efficient manner. In addition, the graphical user interface 12 comprises a second numerical indicator 128 which displays the battery status.

The graphical user interface 12 comprises a routines/macro button 125 which may be adapted for deflecting the bendable section of the elongated surgical device to a specific prestored bent geometric shape. In addition or alternatively, the routines/macro button may be adapted to be operable to transmit an input to the control unit for deflecting the bendable section to at least two different bent geometric shapes in a temporally spaced apart manner. In this case, the control unit is adapted to transmit at least a first and second control command to the actuation unit for deflecting the bendable section to two different bent geometric shapes sequentially in time. This allows to implement routines/macro capabilities which involve execution of a series of predefined commands/bent geometric shapes through a single input, thereby simplifying the operation of the powered steerable system.

FIG. 3B schematically shows a remote control unit 18 of the system which is similarly operable as the graphical user interface 12 in FIG. 3A. The remote control unit 18 comprises a movable control element 181 formed by a slider which has a spatial position 183 that can be adjusted by a user by either moving the movable control element 181 manually, in particular mechanically to receive a haptic feedback. Alternatively or additionally, the control element 181 has control buttons 187 which may be used for an adjustment of the spatial position 183 of the control element 181.

FIGS. 3C to 3E show an elongated surgical device 102 having a straight geometric shape, a first bent geometric shape 41, and second bent geometric shape 43, respectively. The elongated surgical device 102 has a monolithic outer tubular body 7 formed by a nitinol hypotube which has a distal end portion 71 with a bendable section 4. The bendable section 4 has a stress relief section 13 arranged on a lateral side along the bendable section 4 which has a plurality of cutouts which are preferably laser cut into the hypotube. On an opposing side of the bendable section 4, a reinforcement section 14 of the outer tubular body 7 is continuously formed without any cutouts and optionally has further a reinforcing structure such as an increased material thickness of the hypotube. FIG. 3B shows that this stress relief section 13 and reinforcement section 14 allow the bendable section 4 to be deflected to the bent geometric shape 41 if a tension force is applied by an actuation unit (not shown in FIGS. 3A and 3B) to the elongated actuation element 2. In FIGS. 3A and 3B the bendable section is deflectable evenly in a uniform manner along the bendable section which allows to achieve a maximum angular deflection without compromising its structural integrity and durability by reducing localized strain.

The bendable section is further provided with a deflection sensor 9 for determining the shape of the bendable section. The deflection sensor is formed in the specific embodiment as a fiber Bragg grating in a manner known to the skilled person as such. The deflection sensor 9 is connected to a control unit of the system and allows for measuring a parameter indicative of the first and/or second geometric shape 41, 43. The control unit is adapted for determining the first and/or second geometric bent shape 41, 43, compare it with the desired geometric bent shape, and determine a deviation from the desired geometric bent shape. The control unit is further adapted for adjusting the geometric bent shape via operation of the actuation unit until the geometric bent shape 41, 43 approximates the desired geometric bent shape.

FIG. 4 shows a cross-sectional view of a powered steerable system 101 having a decoupling unit 19 in the form of a pair o 4 roller bearings 191, 192 and an elongated surgical device 102 comprising an outer tubular body 7 and an elongated actuation element 2. An actuation unit 3 is operatively connected to a control unit 5 and both are arranged within the common housing 6. The decoupling unit 19 allows for decoupling of a rotational movement of the elongated actuation element 2 from the actuation unit 3 which is attached to an elongated actuation element 2 via the first ball bearings 191. The decoupling unit 19 is further adapted for decoupling the rotational movement of the outer tubular body 7 of the elongated surgical device 102 with a common housing 6 of the powered steerable system 101 via a second ball bearings 192 of the decoupling unit 19.

FIG. 4 shows that the decoupling unit 19 has a synchronized linkage 193 connected to both of the ball bearings 191, 192 which couples the rotational movement of the elongated actuation element 2 and the outer tubular body 7. This allows the elongated actuation element 2 and the outer tubular body 7 to be rotated in unison and enabling a uniform alignment with respect to each other while reducing frictional effects between the elongated actuation element 2 and the tubular body 7.

FIG. 5 shows a schematic view of the various components of the system 101 according to the invention. In particular, FIG. 5 shows that an actuation unit, a processing unit for communication with a force and/or position sensor and for operating the actuation unit, an input interface, and an energy storage device are arranged within a common housing. An elongated actuation element is operable via the actuation unit for bending a bendable section of an elongated surgical device in a previously described manner. The energy storage device powers the actuation unit, the processing unit, and the input interface such that the housing is portable and does not require an external power supply.

A follower unit is connected to the housing of the system 101 for facilitating the operation of the elongated surgical device in a previously described manner in FIGS. 2A and 2B.

The input interface of the system 101 is wirelessly or electrically connected transmission unit. The transmission unit may be powered by the energy storage device as shown in FIG. 5 or connected to an additional energy storage device. The transmission unit in FIG. 5 has a graphical user interface as previously described in FIG. 3A such that user generated inputs can be transmitted to the processing unit and carried out via the actuation unit. As indicated by the dashed arrows the transmission unit may be arranged within the housing and electrically connected to the energy storage and input interface or arranged externally from the housing and wirelessly connected to the input interface.

The FIGS. 6A to 6E show different embodiment of the elongated surgical device 102 having different bendable sections 4. A distal end of the outer tubular body 7 can have different tubular designs which, when subjected to mechanical compression, spatially reconfigure from a straight parent shape to various shapes. These different shapes allow the elongated surgical device 102 formed by a guidewire to help a clinician to access complex anatomic configurations that would normally be close to impossible to access with a classical, non-actively steerable guidewire. Other configurations result in new, unexplored functions to the guidewire, such as, for example, gently anchoring the guidewire into a specific location in a small artery. For instance, as exemplarily shown in FIG. 6A-6E, the elongated surgical device 102 of the invention allows, based on the rational design of its distal end, to obtain some of the most common tip configurations used in interventional neuroradiology, such as for instance so called an angled shape, J-shape, Simon shape, a Cobra shape, or an anchor shape (FIGS. 6A-6E, respectively).

Commercially available devices are delivered out of the package with their tips pre-shaped with these common configurations, as they help the surgeons navigate specific difficult cases. Alternatively, the devices are delivered straight and allow a tip shape remodeling performed by hand by the surgeon. Advantageously, the steerable guidewire of the invention can actively and on-demand change its geometrical configuration when subjected to an actuation

In one embodiment, the elongated surgical device 102 can have its distal end, and particularly its bendable section 4, designed to obtain an “anchor shape” (FIG. 6E) upon actuation. The anchor shape allows the flexible distal end of the elongated surgical device 102 to be curled-up in a 3 dimensional helicoidal shape against the inner wall of an artery. To do so, an elongated reinforcement structure 14 is located in a helicoidal fashion on the distal end and therefore a stress relief section 13, winding about the spatially bendable section 4 (and defining it). The flexibility of the bendable section 4 allows the curled shape to accommodate nearly any artery tortuosity and curve. The main application of this “anchor” is to give the ability to the surgeon to fix the guidewire's tip at a specific location and create a so-called “fixed point” which will facilitate the insertion of catheter devices on top of the established guiding wire and avoid unwanted movement (slippage) of the guidewire. FIGS. 7A and 7B show a schematic view of two different embodiments of an actuation unit 3 of the system 101.

FIG. 7A shows a schematic view of a rotary actuation unit 3 formed by a rotating motor and a pulley. The rotating motor is connected to an elongated actuation element 2 for deflecting the bendable section of the elongated surgical device 102 in a previously described manner. An outer tubular body 7 of the elongated surgical device 102 is connected to a housing of the system 101. This enables a safe and kink-free storage of the elongated actuation element 2 by the actuation element 2 being wound/unwound on the pulley via the rotating motor and further enables a particularly compact design of the steerable system.

FIG. 7B shows a schematic view of an actuation unit formed by a linear motor. The linear motor allows for a simple design without any backlash and a spatial position of the elongated actuation element 2 with respect to an outer tubular body 7/a housing 6. The spatial position of the elongated actuation element 2 may be determined by a positional sensor, e.g. a Hall sensor, in a reliable manner (see FIGS. 10A and 10B). FIGS. 8A to 8D show different embodiments of a follower unit 8 of the system 101. The follower unit 8 is formed by a low-friction cylindrical sleeve having an inner conduit with a side-wards and a distal opening such that a housing 6 of the system 101 can move in a rotational direction R and a longitudinal direction L within the conduit of the follower unit 8. An internal surface of the follower unit 8 may be formed or coated by a material having a low coefficient of friction, such as polytetrafluoroethylene, polyoxymethylene, polyamide, or high molecular weight polyethylene. This allows the follower unit 8 to provide a low and constant frictional resistance to facilitate a movement in the rotational direction R and in the longitudinal direction L of the elongated surgical instrument 102 with respect to the follower unit 8.

The follower unit 8 in FIG. 8B has a biasing member 83, in particular a spring, which connects a common housing 6 of the system 101 to a proximal end of the follower unit 8. The biasing member 83 may be adapted for providing a predefined frictional resistance and haptic feedback allowing for precise fine adjustment of the rotational and translational position of the common housing 6 with respect to the follower unit 8. In addition or alternative, the biasing member 83 may be adapted to bias the common housing 6 to a predefined spatial position such that a restoring force of the biasing member 83 facilitates operation, especially manual operation of the elongated surgical instrument 102.

The follower unit 8 in FIG. 8C has a follower drive 85 comprising a translational actuator which is adapted for moving the common housing 6 with respect to the follower unit 8, e.g. by a nut engaging a threaded shaft connected to the housing 6 to cause linear displacement of the common housing 6.

The follower drive 85 is uncoupled, e.g. via a swivel coupling mechanism, from a rotational movement of the common housing 6 in the rotational direction R such that the elongated surgical device 102 may still be manually rotatable with respect to the follower drive 85.

The follower unit 8 has a force/position sensor 82 which is adapted for detecting force or position data indicative of the translational actuation of the elongated surgical device 102 and the housing 6. The force/position sensor 82 is connected to a control unit (not shown in FIG. 8C). The control unit is connected to a follower drive 85 of the follower unit 8 and configured for processing the force/position data in real-time. The control unit is configured to operate the follower drive 85 based on the force/position data such that the housing 6 and the elongated surgical device 102 may be moved in a synchronized manner in the direction of the detected manual actuation of the elongated surgical device 102. This allows facilitated manual movement of the elongated surgical device 102 by a user by supporting the translational movement of the elongated surgical device 102.

The control unit may further comprise a dynamic actuation modulation mechanism which is adapted to interpret a degree of the user actuation and adjusts the actuation of the follower drive 85 based on the degree of the user actuation. This may allow for a slower and more exact fine adjustment of translational position of the elongated surgical device 102 and housing 6 for weaker user actuation and faster coarse adjustment for higher measured actuation values.

The follower drive 85 may have an encoder recording the elongated surgical device 102 and the common housing 6. The encoder and the control unit may further be configured for providing the user with the exact rotational and/or translational spatial position of the housing 6, e.g. by wirelessly communication to a wireless transmission unit (see FIGS. 2A and 2B).

In an alternative embodiment (not shown in FIGS. 8A-8D), the follower drive 85 may further be adapted for rotating the elongated surgical device 102 and the common housing 6 together with respect to the follower unit 8 analogously to the translational movement.

FIG. 8D shows low-friction elements 84 which are circumferentially arranged around the housing 6 and allow a more precise and low-friction movement within the cylindrical sleeve of the follower unit 8 in the translational direction L and the rotational direction R.

FIGS. 9A and 9B show a schematic view of a first and second embodiment of the powered steerable system 101 which has a housing 6 coupled to a follower unit 8 which is formed by suspension unit 63 connected to a solid support 104 of the system 101. The suspension unit 63 of the follower unit 8 is connected at a connection point to the housing 6 and the solid support 104 respectively and is adjustable in length to allow for a translational movement of the housing having a similar effect as a previously described follower units (see FIGS. 8A-8D). This allows for a more controlled movement of the housing 6 by reducing/compensating a frictional resistance/an inertia of the housing 6 and at the same time prevents unintentional accidental movement of the housing 6 by the suspension unit 63 being coupled to the solid support 104.

The housing 6 in FIGS. 9A and 9B is rotatably connected, e.g. via a swivel coupling mechanism 61, e.g. at the connection point to the housing, to the suspension unit 63 of the follower unit 8 and the solid support 104 such that the housing 6 and an elongated surgical device 102 are rotatable, in particular manually rotatable, in unison in a rotational direction R. In addition, the housing 6 and the elongated surgical device 102 are translationally movable in a longitudinal direction L by extending/retracting the suspension unit 63 of the follower unit 8. The suspension unit 63 in FIG. 9A may be formed by a spring-loaded pulley such that it may manually adjusted by the clinician and passively retains its adjusted spatial position and orientation.

FIG. 9B shows that the follower unit 8 of the system 101 may have an off-axis rotational coupling 62 which is adapted such that a longitudinal axis of the housing 6 and elongated surgical device 101 may be laterally rotated with respect to the solid support 104. This may be achieved, by the suspension unit 63 of the follower unit 8 being rotatable around the connection point to the solid support 104 to adjust the off-axis rotation angle of the housing 6. This allows a clinician to adjust the position of the housing 6 and elongated surgical device 101 in an additional rotational degree of freedom to facilitate the alignment and operation of the system 101 according to the clinical needs.

The follower unit 8 of FIGS. 9A and 9B may comprise at least one actuator, in particular a motorized pulley, which may be configured operate the translational movement in the longitudinal direction, the rotational movement of the elongated surgical device 102 and in particular the housing 6, and/or the off-axis rotation of the housing 6/elongated surgical device 102.

FIGS. 10A and 10B show a schematic view of two embodiments of the powered steerable system 101 which have two different deflection sensors 9 for measuring the displacement of the elongated actuation element 2, e.g. formed by a bidirectional actuation rod, of an elongated surgical device 102. The systems 101 in FIGS. 10A and 10B have a control unit 5, an actuation unit 3, and a deflection sensor 9 which are arranged within a common housing 6.

The elongated surgical instrument 102 has an outer tubular body 7 which may be coupled to the elongated actuation element 2 via a previously described synchronized linkage 193 indicated by the dashed lines such that they are movable in unison in a rotational direction R (see FIG. 4). A rotational movement of the outer tubular body 7 and the elongated actuation element 2 is uncoupled from the housing 6 and the actuation unit 3 by a decoupling unit 19, e.g. by having two annular ball bearings 191, 192.

The control unit 5 is adapted for operating the actuation unit 3 and receiving deflection data from the deflection sensor 9 indicative of the position of a proximal end of the elongated actuation element 2 along a longitudinal direction L. The control unit is configured for determining the geometric shape of a bendable section of the elongated surgical instrument 102 based on the detected deflection data.

The deflection sensor 9 in FIG. 10A is formed by a resistive deflection sensor which is arranged in a distal tip of a shaft of the actuation unit 3 allowing for a simple design.

The deflection sensor 9 in FIG. 10B is formed by a Hall sensor which measures deflection data in form of a displacement of a magnetic field inducer 91 which is connected to the shaft of the actuation unit 3.

FIG. 11 shows a remote control unit 18 according to the invention mounted on a catheter valve 201 of a catheter 20. The system 101 may comprise the catheter 20 and preferably the remote control unit 18 mountable to a catheter 20. Mounting the remote control unit 18 on the catheter 20 allows the clinician to have the remote control unit 18 readily available and visible when carrying out a surgical intervention.

The remote control unit 18 is designed as previously described in FIG. 3B and is operable by control buttons 187 for adjustment of a spatial position 183 of a control element 181. The remote control unit 18 is further adapted for wirelessly transmitting a user input, such as operation of the control buttons 187, to a control unit of the system (not shown in FIG. 11) such that the elongated surgical device 102 can be deflected to a desired geometric shape. FIG. 11 shows that the elongated surgical device 102 is insertable through the catheter 20 of the system which may be specifically designed for receiving the elongated surgical device 102 within its inner lumen to promote a seamless interaction.

1.-23. (canceled)

24. A powered steerable system for navigating an elongated surgical device having an outer tubular body and an elongated actuation element nested within the outer tubular body through a bodily lumen of a patient, the steerable system comprising:
(a) an actuation unit configured to be coupled to the elongated actuation element for actuating the elongated actuation element;
(b) an input interface which is configured for receiving an input specifying a deformed geometric shape of a bendable section at a distal end portion of the outer tubular body;
(c) a control unit operatively coupled to the input interface and configured for generating a control command based on the input; and
(d) an output interface operatively coupled to the control unit configured for transmitting the control command to the actuation unit, wherein
the actuation unit is adapted to deform the bendable section to the deformed geometric shape based on the control command.

25. The powered steerable system according to claim 24, wherein the actuation unit is configured to generate movement of the elongated actuation element in both a proximal-directed and distal-directed linear movement and the actuation unit is formed by
(i) a linear actuation unit or
(ii) a rotary actuation unit which comprises a conversion mechanism operatively connected or connectable to the elongated actuation element, wherein the conversion mechanism is adapted to translate the rotary force of the rotary actuation unit into a linear movement of the elongated actuation element.

26. The powered steerable system according to claim 24, wherein the control unit and the actuation unit are positioned within a common housing of the powered steerable system.

27. The powered steerable system according to claim 26, wherein the common housing has a longitudinal dimension within a range of 0.1 cm to 25 cm and a lateral dimension within a range of 0.036 cm to 10 cm.

28. The powered steerable system according to claim 24, wherein the powered steerable system has a positioning unit which is controllably coupled to the control unit and is configured for at least one of:
(a) rotating the elongated surgical device around the central trajectory of the elongated surgical device and
(b) translationally moving the elongated surgical device in a distal or proximal direction, based on a movement input.

29. The powered steerable system according to claim 24, wherein the powered steerable system has a follower unit which has
(a) at least one bearing member which is configured for supporting the actuation unit while moving the actuation unit in at least one of the distal, the proximal, and a rotational direction around a central trajectory of the actuation unit, when moving the elongated surgical device, or
(b) a sensor which is configured to generate force or position data by detecting a rotational or translational actuation of the elongated surgical device and a follower drive which is adapted to move the actuation unit along at least one of the translational and the rotational direction in a synchronized manner with the elongated surgical device based on the force or position data in real-time.

30. The powered steerable system according to claim 24, wherein the control unit is configured for applying vibrations via the actuation unit or the positioning unit to the elongated surgical device at a frequency between 1 Hz-1000 Hz when deflecting the bendable section.

31. The powered steerable system according to claim 24, wherein the powered steerable system has at least one deflection sensor configured to generate deflection data by detecting at least one of a position of the elongated actuation element and a geometric shape of the bendable section, and the control unit is configured
(a) to receive the deflection data from the deflection sensor,
(b) to determine if the desired geometric shape is achieved based on the deflection data, and
(c) to control the actuation unit based on the detected deflection data in a closed-feedback loop to achieve the desired geometric shape of the bendable section in real-time.

32. The powered steerable system according to claim 31, wherein the at least one deflection sensor is selected from at least one of a magnetic deflection sensor, an optical deflection sensor, a capacitive deflection sensor, and a resistive deflection sensor.

33. The powered steerable system according to claim 24, wherein the powered steerable system comprises an energy storage device for operating the steerable system.

34. The powered steerable system according to claim 24, wherein the system comprises a graphical user interface, wherein the graphical user interface is configured for at least one of:
(a) to present visual information on a display,
(b) to provide a movable control element on the display which has a spatial position that can be adjusted by a user to adjust a degree of the bent geometric shape,
(c) to provide at least one of a numerical and graphical indicator functionally coupled to the movable control element which displays the degree of the bent geometric shape in real time, and
(d) to transmit an input based on the spatial position of the movable control element to the input interface of the control unit specifying the bent geometric shape in real time.

35. A computer implemented method for navigating an elongated surgical device having an outer tubular body and an elongated actuation element nested within the outer tubular body through a bodily lumen of a patient, the method comprising the steps:
(a) receiving an input via an input interface specifying a desired geometric shape of a bendable section at a distal end portion of the outer tubular body;
(b) calculating a control command based on the input via a control unit;
(c) transmitting the control command to an actuation unit via an output interface; and
(d) deflecting the bendable section to the desired geometric shape based on the control command.

36. A non-transitory computer-readable medium comprising instructions that, when executed by a processor of a powered steerable system, cause the powered steerable system of claim 1 to perform the method of claim 35.

37. The powered steerable system according to claim 24, wherein the steerable system comprises the elongated surgical device having the outer tubular body and at least one of the tension responsive elongated actuation element and the compression responsive elongated actuation element nested within the outer tubular body, wherein the distal end portion of the outer tubular body has the bendable section which is deformable to the bent geometric shape.

38. The powered steerable system according to claim 37, wherein the bendable section is uniformly laterally deflectable along its longitudinal length and the bent geometric shape is defined by a uniform lateral bending angle over the entire bendable section, wherein the control unit and the actuation unit are configured such that the bending angle is adjustable over the entire range from 0° to 540°.

39. The powered steerable system according to claim 37, wherein one lateral side of the bendable section of the elongated surgical device has a stress relief section so that the bendable section is deflected in a lateral direction of the stress relief section when at least one of the tension force and the compression force is applied to the elongated actuation element.

40. The powered steerable system according to claim 37, wherein one lateral side of the bendable section of the elongated surgical device has a reinforcement portion which is longitudinally rigid, such that applying at least one of the tension force and the compression force to the elongated actuation element essentially does not longitudinally affect the length of the reinforcement portion.

41. The powered steerable system according to claim 37, wherein a distal end of the elongated actuation element is connected to a terminal distal tip of the elongated surgical device, and a proximal end of the elongated actuation element is longitudinally movably connected to the actuation unit and a proximal end of the outer tubular body is longitudinally immovably connected with respect to the actuation unit.

42. The powered steerable system according to claim 37, wherein the elongated surgical device is sized and shaped for use in peripheral interventions, in interventional cardiology, or in neurovascular surgical procedures, wherein the outer tubular body has a maximum cross-sectional dimension of less than 1 mm.

43. The powered steerable system according to claim 37, wherein the outer tubular body is formed by a monolithic tubular element.
